(19) United States
(12) Patent Application Publication
(10) Pub. No.: US 2014/0341865 A1
(43) Pub. Date: Nov. 20, 2014

(54) Dressing Material With Cell Components For Wound Healing

(71) Applicant: Tego Science Inc., Seoul (KR)

(72) Inventors: Saewha Jeon, Seoul (KR); Yun Hee Kim, Seoul (KR); Ho Yun Chung, Daegu (KR); Jin Hyun Choi, Daegu (KR); Hankyu Jang, Incheon (KR)

(73) Assignee: TEGO SCIENCE INC., Seoul (KR)

(21) Appl. No.: 14/364,890

(22) PCT Filed: Dec. 12, 2012
(86) PCT No.: PCT/KR2012/010800

§ 371 Date: Jun. 12, 2014

(30) Foreign Application Priority Data
Dec. 12, 2011 (KR) 10-2011-0132867

(51) Int. Cl. A61L 15/00

(52) U.S. Cl. CPC A61L 15/00; A61L 2300/412; A61L 2430/34
USPC 424/93.7; 435/180; 602/48

(57) ABSTRACT

There is provided a dressing for treating a wound. The dressing for healing a wound can be useful in maintaining a moisture environment at a wound site using a biocompatible polymer scaffold, and effectively promoting healing of a wound by various growth factors secreted by skin cells or stem cells attached to the biocompatible polymer scaffold as well.

### BACKGROUND

The present disclosure relates to a dressing for treating a wound.

Dressing refers to a procedure conducted to cover a wound site to protect the wound site, that is, a procedure conducted to cover, support or fix a wound site with sterilized gauze bandage, and the like. Such dressing serves to suppress bleeding, prevent infection of a wound site and interrupt the spread of affected parts. In the year 1962, Winder reported that wound healing in the skin was excellent under a moisture environment. Since then, many studies were reported to verify the wound healing effect. In recent years, in the case of methods for healing a wound, conventional methods for dressing a wound site with gauze under a dry environment have been rapidly replaced with methods for dressing a wound site under a moisture environment.

To dress a wound site under such a moisture environment, much research on dressings for treating a wound using a biocompatible polymer has been conducted. Various kinds of dressings using a biocompatible polymer have already been on the market. However, the dressings for treating a wound using the biocompatible polymer have problems in that these dressings themselves do not have a wound healing activity since the dressings simply focus on maintaining a moisture environment.

Therefore, development of new dressings capable of preventing the spread of damaged wound site, providing a moisture environment and promoting healing of a wound as well is required.

### SUMMARY

An aspect of the present disclosure may provide a novel biological dressing capable of maintaining a moisture environment at a wound site and promoting healing of a wound since the dressing itself has a tissue regenerating function, and a method for preparing the same.

According to an aspect of the present disclosure, a dressing may include a biocompatible polymer scaffold, and skin cells or stem cells attached to the biocompatible polymer scaffold.

According to another aspect of the present disclosure, a method of preparing a dressing may include coating a biocompatible polymer scaffold with a cell-adhesive polymer, and attaching skin cells or stem cells to the biocompatible polymer scaffold coated with the cell-adhesive polymer.

According to still another aspect of the present disclosure, a method of preparing a dressing may include attaching skin cells or stem cells to a biocompatible polymer scaffold, and coating the biocompatible polymer scaffold having the skin cells or stem cells attached thereto with a cell-adhesive polymer.

According to still another aspect of the present disclosure, a method of preparing a dressing may include preparing a mixed solution by mixing skin cells or stem cells with a cell-adhesive polymer, and attaching the mixed solution to a biocompatible polymer scaffold.

According to yet another aspect of the present disclosure, a method of preparing a dressing may further include culturing the skin cells or stem cells attached to the biocompatible polymer scaffold.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a diagram showing a structure of a dressing for treating a wound according to one exemplary embodiment of the present disclosure;

FIG. 2 is a diagram showing the results obtained by observing keratinocytes on a surface of a contact layer of a dressing forming a complex with keratinocytes under a scanning electron microscope;

FIG. 3 is a diagram showing the results obtained by observing keratinocytes in the dressing forming a complex with keratinocytes under a scanning electron microscope, a uplight microscope (H&E stain) and a fluorescence microscope (DAPI stain);

FIG. 4 is a diagram showing the results obtained by determining characteristics of the keratinocytes used in the dressing according to one exemplary embodiment of the present disclosure using an immunofluorescence staining;

FIG. 5 is a diagram showing the results obtained by measuring amounts of wound healing-associated proteins expressed in the keratinocytes used in the dressing according to one exemplary embodiment of the present disclosure using an enzyme-linked immunosorbent assay;

FIG. 6 is a diagram showing the results obtained by measuring a wound healing rate of the dressing according to one exemplary embodiment of the present disclosure; and

FIG. 7 is a diagram showing the results obtained by determining in vivo wound healing effect of the dressing according to one exemplary embodiment of the present disclosure through histological analysis.

### DETAILED DESCRIPTION

Exemplary embodiments of the present disclosure will now be described in detail with reference to the accompanying drawings.

The disclosure may, however, be exemplified in many different forms and should not be construed as being limited to the specific embodiments set forth herein. Rather, these embodiments are provided so that this disclosure will be thorough and complete, and will fully convey the scope of the disclosure to those skilled in the art.

In the drawings, the shapes and dimensions of elements may be exaggerated for clarity, and the same reference numerals will be used throughout to designate the same or like elements.

The present disclosure is directed to a dressing including a biocompatible polymer scaffold, and cells or stem cells attached to the biocompatible polymer scaffold.

In the present disclosure, “attachment of cells or stem cells” to a biocompatible polymer scaffold may encompass coating a surface of the biocompatible polymer with the skin cells or stem cells, or injecting the skin cells or stem cells into the biocompatible polymer scaffold.

In general, a wound (cut) healing procedure is divided into an inflammatory stage, a proliferative stage, and a mature stage. When blood vessels are injured due to damage of tissues in the inflammatory stage, many kinds of growth factors (PDGF, TGF-β, EGF, FGF, and the like) and cytokines (IL-1, IL-6, IL-8, TNF, and the like) are released from platelets and inflammatory cells at a bleeding site, and epithelial cells spread on a surface of a wound to cover the wound. In the proliferative stage, such growth factors and cytokines promote the growth of endothelial cells, fibroblasts, epidermal cells, and the like. In the mature stage, the grown cells themselves release growth factors to form granulation tissues, and collagen fibers (III) or elastic fibers are produced. Then, a wound healing is completed after these cells undergo a tissue reconstruction stage. In the above-describe healing procedure, conditions such as moisture and non-infection environments in which epithelial cells can be easily swarmed, no foreign substances and necrotic tissues, and high-concentration of cell growth factors are required to effectively heal a wound.

The dressing according to one exemplary embodiment of the present disclosure has effects of maintaining a moisture environment at a wound site using the biocompatible polymer scaffold, and effectively promoting healing of a wound through tissue regeneration by releasing various kinds of growth factors (TGF-α, VEGF, FGF, EGF, MMP-2 and MMP-9, and the like) and cytokines (IL-1α, and the like) from the skin cells or stem cells attached to the biocompatible polymer scaffold as well.

In the present disclosure, the term “biocompatible polymer scaffold” refers to a scaffold including a biocompatible polymer, that is, a structure based on a dressing having skin cells or stem cells attached thereto and providing a contact surface with a wound site. The biocompatible polymer scaffold may promote a wound healing effect by a complex function of the release of biological factors and the formation of a proper moisture environment by interfering with the influx of foreign substances and releasing or storing an exudate at a wound site to maintain a proper moisture environment.

In the present disclosure, the term “biocompatible polymer” refers to a polymer material which is not harmful to a human body, that is, a synthetic or natural polymer material which does not release substances harmful to a human body and cause side effects such as skin stimulation even when coming in direct contact with cells and a wound site, and have a negative influence on the human body. Any polymer materials known to be usable as a dressing material may be used as the biocompatible polymer without limitation, and may be properly chosen by those skilled in the related art. The biocompatible polymer may, for example, include at least one selected from the group consisting of polyvinyl alcohol (PVA), polyurethane (PU), polyethylene (PE), polyacrylic acid (PAA), polyoxyethylene (POE), polyethylene oxide (PEO), polytetrafluoroethylene (PTFE), polypropylene (PP), polyethylene terephthalate (PET), polyamide (PA), polyacrylonitrile (PAN), polyester (PES), polyvinyl chloride (PVC), polyvinylidenefluoride (PVDF), polysiloxane (a silicone rubber), polyglycolic acid (PGA), polylactic acid (PLA), polymethacrylic acid (PMA), polyacrylamide (PAM), polysaccaride (PS), polyvinylpyrrolidone (PVP), silicone, alginic acid, sodium alginate, cellulose, pectin, chitin, chitosan, gelatin, collagen, fibrin, hyaluronic acid, natural rubber, and synthetic rubber, but the present disclosure is not limited thereto.

The biocompatible polymer scaffold may be prepared by gathering the biocompatible polymer in a cotton shape and processing the biocompatible polymer in a sheet shape, or may be prepared in the form of a non-woven fabric, woven fabric or cloth which is formed of a biocompatible polymer. In addition, the biocompatible polymer scaffold may be used in the form of a film, foam, hydrocolloid, hydrogel, or non-woven fabric, but the present disclosure is not limited thereto. For example, the biocompatible polymer scaffold may be properly processed and used by those skilled in the related art.

In the present disclosure, the term “skin cells” refers to cells forming the skin (the epidermis, the dermis, and the subcutaneous tissue). In this case, the kinds of the skin cells are not particularly limited. For example, the skin cells may be keratinocytes and melanocytes which are present in the epidermis, and fibroblasts, endothelial cells and hair follicle stem cells which are present in the dermis and take part in biosynthesis of collagen and elastin.

In the present disclosure, the kinds of the stem cells are not particularly limited, but may, for example, be embryonic stem cells or adult stem cells. The “embryonic stem cells” include all kinds of embryonic stem cells derived from mammals. For example, the embryonic stem cells may be human embryonic stem cells. The “adult stem cells” refer to stem cells derived from a skin, a liver, a lung, blood, a bone marrow, a fat, an amnion, an endometrial tissue, and cord blood of an adult, that is, cells that can differentiate into all kinds of tissues.

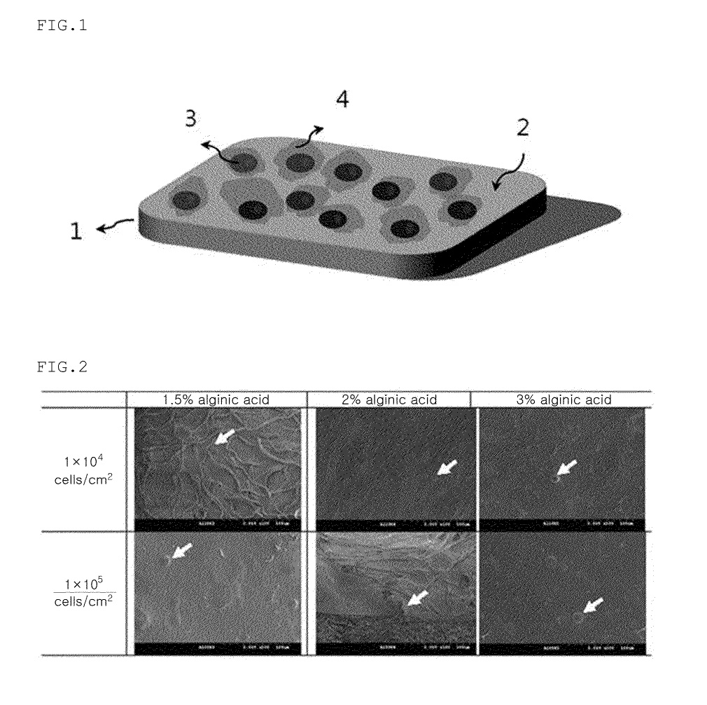

FIG. 1 is a diagram showing a dressing according to one exemplary embodiment of the present disclosure. Referring to FIG. 1, since skin cells or stem cells 3 are attached to a biocompatible polymer scaffold 1, the dressing according to one exemplary embodiment of the present disclosure may promote a wound healing effect by releasing cytokines or growth factors from the skin cells or stem cells 3 attached to the biocompatible polymer scaffold 1, and exposing the cytokines or growth factors 4 onto a wound contact surface 2 when the dried dressing for treating a wound runs into an exudate in a wound.

Also, the dressing according to one exemplary embodiment of the present disclosure may be obtained by coating a biocompatible polymer scaffold or skin cells or stem cells attached to the biocompatible polymer scaffold with a cell-adhesive polymer.

According to one exemplary embodiment of the present disclosure, the biocompatible polymer scaffold may be coated with a cell-adhesive polymer. That is, the dressing according to one exemplary embodiment of the present disclosure may be obtained by attaching the skin cells or stem cells to the biocompatible polymer scaffold coated with the cell-adhesive polymer.

According to another exemplary embodiment of the present disclosure, the skin cells or stem cells attached to the biocompatible polymer scaffold may be coated with the cell-adhesive polymer. That is, the dressing according to another exemplary embodiment of the present disclosure may be attached to the biocompatible polymer scaffold in a state where the skin cells or stem cells are coated with the cell-adhesive polymer.

According to still another exemplary embodiment of the present disclosure, at least one surface of the biocompatible polymer scaffold having the skin cells or stem cells attached thereto may be coated with the cell-adhesive polymer.

In the present disclosure, the term “cell-adhesive polymer” refers to a polymer material supporting cell attachment. For example, the cell-adhesive polymer may provide adhesivity so that the skin cells or stem cells can be readily fixed in the biocompatible polymer scaffold, and also provide adhesivity so that the dressing according to one exemplary embodiment of the present disclosure coated with the cell-adhesive polymer can be attached to skin cells at a wound site. For example, the cell-adhesive polymer may be alginic acid, fibrin, gelatin, collagen, or hyaluronic acid, but the present disclosure is not limited thereto. For example, the cell-adhesive polymer may be properly chosen by those skilled in the related art. Also, the cell-adhesive polymer may be used in the form of an aqueous solution or hydrogel.

According to still another exemplary embodiment of the present disclosure, the skin cells or stem cells may be cultured cells. That is, in the dressing according to one exemplary embodiment of the present disclosure, the skin cells or stem cells may be attached to the biocompatible polymer scaffold, and then cultured. More particularly, in the dressing according to one exemplary embodiment of the present disclosure, the skin cells or stem cells may be attached to the biocompatible polymer scaffold coated with the cell-adhesive polymer, and then cultured.

According to yet another exemplary embodiment of the present disclosure, at least one surface of the biocompatible polymer scaffold having the skin cells or stem cells attached thereto may be coated with the cell-adhesive polymer, and the skin cells or stem cells may then be cultured.

In this case, a medium used to culture the skin cells or stem cells according to one exemplary embodiment of the present disclosure may include any media for culturing animal cells as known in the related art, including DMEM, F12, RPMI1640, MEM, DMEM/F12, and a serum-free medium (SFM).

Also, the present disclosure is directed to a method for preparing a dressing, which may include coating a biocompatible polymer scaffold with a cell-adhesive polymer, and attaching skin cells or stem cells to the biocompatible polymer scaffold coated with the cell-adhesive polymer. Also, the method for preparing a dressing may further include culturing the skin cells or stem cells attached to the biocompatible polymer scaffold.

In addition, the present disclosure is directed to a method for preparing a dressing, which may include attaching skin cells or stem cells to a biocompatible polymer scaffold, and coating the biocompatible polymer scaffold having the skin cells or stem cells attached thereto with a cell-adhesive polymer. Also, the method for preparing a dressing may further include culturing the skin cells or stem cells after the biocompatible polymer scaffold is coated with the cell-adhesive polymer.

Further, the present disclosure is directed to a method for preparing a dressing, which may include preparing a mixed solution by mixing skin cells or stem cells with a cell-adhesive polymer, and attaching the mixed solution to a biocompatible polymer scaffold. Also, the method for preparing a dressing may further include culturing the skin cells or stem cells after the mixed solution is attached to the polymer scaffold.

In the method for preparing a dressing according to one exemplary embodiment of the present disclosure, the attaching of the skin cells or stem cells to the biocompatible polymer scaffold may be performed by coating a surface of the biocompatible polymer scaffold with the skin cells or stem cells or injecting the skin cells or stem cells into the biocompatible polymer scaffold. Here, the injection may be performed by introducing the mixed solution of cells and a medium for culturing animal cells into the biocompatible polymer scaffold.

The characteristics of the biocompatible polymer scaffold, the cell-adhesive polymer and the skin cells or stem cells used herein are as described above.

In the method for preparing a dressing according to another exemplary embodiment of the present disclosure, the coating of the biocompatible polymer scaffold or the biocompatible polymer scaffold having the skin cells or stem cells attached thereto with the cell-adhesive polymer may be performed by attaching the cell-adhesive polymer in the form of an aqueous solution or hydrogel.

In the method for preparing a dressing according to still another exemplary embodiment of the present disclosure, the preparing of the mixed solution by mixing the skin cells or stem cells with the cell-adhesive polymer may be performed by mixing the cell-adhesive polymer in the form of an aqueous solution or hydrogel with the skin cells or stem cells.

Hereinafter, the present disclosure will be described in further detail with reference to Preparative Examples and Experimental Examples. However, it should be understood that detailed description provided herein is merely intended to provide a better understanding of the present disclosure, but is not intended to limit the scope of the present disclosure, as apparent to those skilled in the art.

### Example 1

### Preparation of Dressing Forming Complex with Keratinocytes Using Coating Method

Keratinocytes (Skin Bank TG004, Tego Science Inc.) were attached to an alginic acid non-woven fabric having a square shape with a size of 2 cm×2 cm in a density of 1×10⁴ or 1×10⁵ per 1 cm². The cell attachment was performed by coating the keratinocytes with aqueous alginate solution, and the aqueous alginate solution was prepared by dissolving alginic acid in distilled water at each concentration of 0.5%, 1%, 1.5%, 2%, and 3% and sterilizing through a filter with a pore size of 0.22 μm. To coat an alginic acid non-woven fabric having an area of 4 cm² with cells, a total of 4×10⁴ or 4×10⁵ keratinocytes were counted, and centrifuged to form a cell pellet. Thereafter, the cell pellet was mixed with 50 μl of physiological saline solution. The cell mixture was mixed with 500 μl of the aqueous alginate solution at each concentration to form a total of 550 μl of the mixture solution. Then, the alginic acid non-woven fabric having an area of 4 cm² was coated with 550 μl of the mixture solution. As the control, the alginic acid non-woven fabric having an area of 4 cm² was coated with 550 μl of a mixture solution obtained by mixing 500 μl of an aqueous alginate solution with 50 μl of a physiological saline solution without cells. Subsequently, the alginic acid non-woven fabrics were dried for 7 days in a vacuum dry oven, and then reacted in 0.2% CaCl₂ for 24 hours to promote a cross-linking reaction of alginic acid. The alginic acid non-woven fabrics were immersed in distilled water, washed for 24 hours, and dried for 7 days in a vacuum dry oven to prepare a dressing forming a complex with keratinocytes.

### Example 2

### Preparation of Dressing Forming Complex with Keratinocytes Using Injection Method

Keratinocytes (Skin Bank TG004, Tego Science Inc.) were attached to a collagen sponge having a square shape with a size of 2 cm×2 cm in a density of 1×10⁴ per 1 cm². The cell attachment was performed by mixing and diluting a total of 4×10⁴ keratinocytes with 100 μl of a DMEM/F12 medium, pipetting the cells, and injecting the mixed solution into the sponge. The cell-injected sponge was cultured at 37° C. for 7 days in a DMEM/F12 medium supplemented with an epidermal growth factor (EGF) at a concentration of 10 ng/ml and 10% FBS, and lyophilized to prepare a dressing forming a complex with keratinocytes.

### Experimental Example 1

### Experiment on Effect of Dressing Forming Complex with Keratinocytes

### 1-1. Determination of Presence of Cells

To determine whether cells were distributed on a surface of the dressing forming a complex with keratinocytes prepared in Example 1, the dressing was fixed for several hours in a fixing solution including 2.0% paraformaldehyde (pH 7.4), and a surface of the dressing was observed under a scanning electron microscope (SEM) to determine the presence of the cells.

As a result, as shown in FIG. 2, it was confirmed that the keratinocytes (arrows) were present on a surface of a contact layer in the dressing in which 1×10⁴ and 1×10⁵ keratin cells were coated with each of the aqueous alginate solutions having concentrations of 1.5%, 2% and 3% (see FIG. 2).

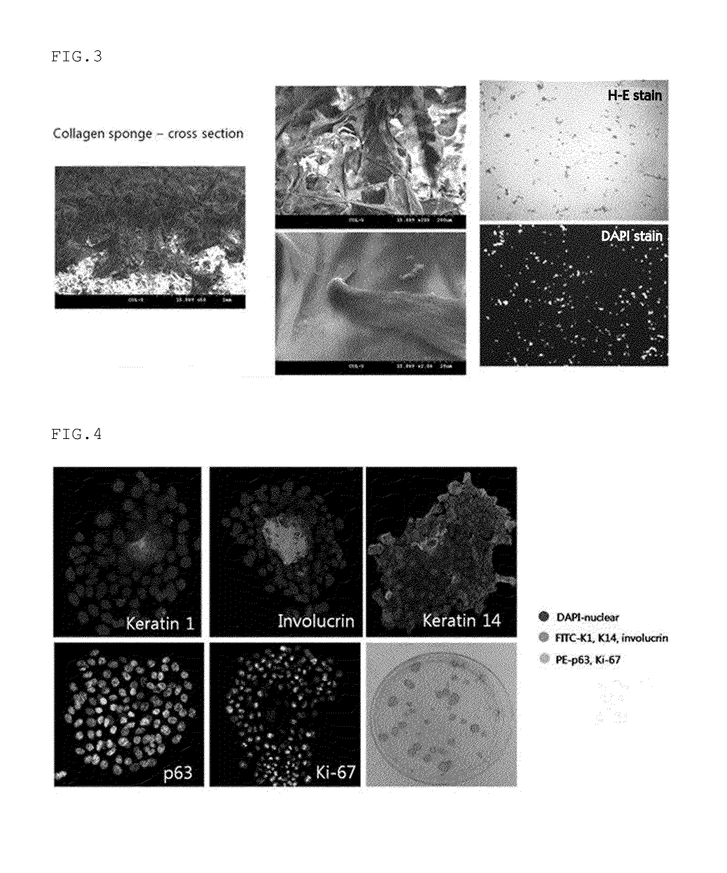

To determine whether cells were distributed in the dressing forming a complex with keratinocytes prepared in Example 2, the dressing was cut, and a cut surface of the dressing was stained for 15 minutes in 2 μg/ml of a DAPI (4′,6-diamidino-2-phenylindole) solution. After the staining, the cut surface of the dressing was observed under a fluorescence microscope, and histological analysis showed that the cells were present in the dressing using a Hematoxylin-Eosin staining method (see FIG. 3).

### 1-2. Characteristics of Keratinocytes

To analyze characteristics of the keratinocytes used for preparation of the dressings in Examples 1 and 2, expression of specific proteins was observed using an immunofluorescence staining method. Keratinocytes co-cultured with 3T3 cells for days in a culture medium supplemented with 10% fetal bovine serum were fixed for several minutes in a fixing solution (methanol:acetone=1:1), reacted with each primary antibody against specific markers of kerationcytes (Keratin 1, Keratin 14, or Involucrin), proliferating cells (Ki-67), and stem cells (P63), and then reacted with secondary antibodies conjugated with fluorescent dye. The nuclei of the cells were stained with DAPI.

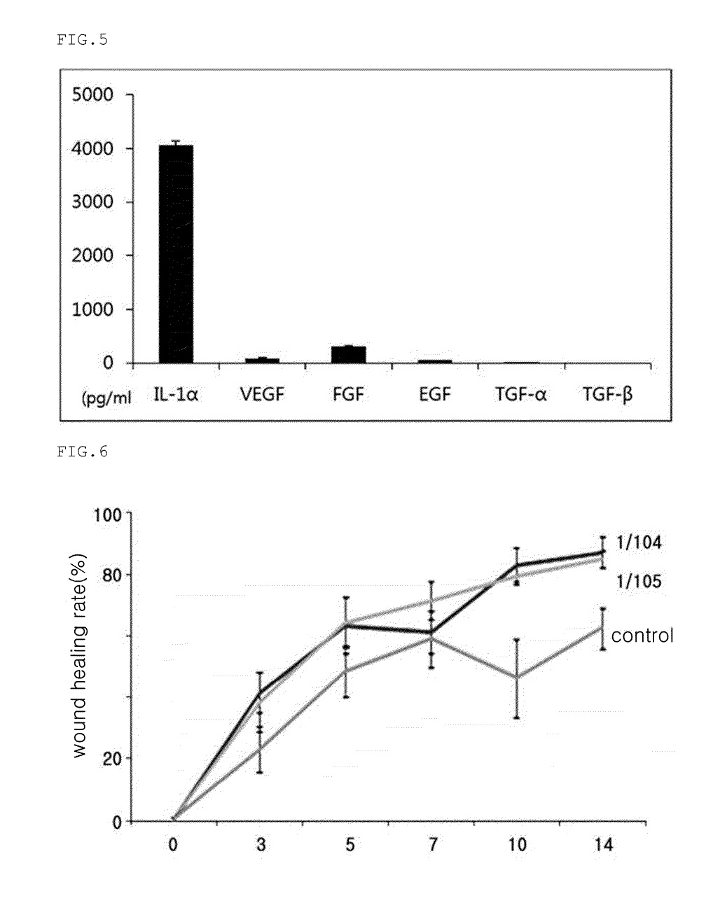

As a result, as shown in FIG. 4, it could be seen that keratin 14, Ki-67 and p63 were expressed in the keratinocytes simultaneously with expression of Keratin 1 and Involucrin, and thus keratinocytes showed colony forming activity that was a characteristic of the stem cells, indicating that the keratinocytes had a high growth rate and exhibited the characteristics of the stem cells (see FIG. 4).

1-3. Measurement of Amount of Proteins Associated with Wound Healing

To evaluate efficacy of the dressing forming a complex with keratinocytes prepared in Example 1, amounts of proteins associated with the wound healing were measured. A portion of the cell extract separated in Example 1 was taken, and expression levels of the wound healing-associated proteins, that is, a cytokine (IL-1 alpha) and growth factors (TGF-alpha, VEGF, FGF, MMP-2 and MMP-9) were quantified using an enzyme-linked immunosorbent assay (ELISA). In an experimental method, a kit for enzyme-linked immunosorbent assays commercially available for each protein was used, and the proteins were measured and quantified according to the manufacturer's protocol of the kit. That is, 100 μl of a solution of proteins extracted from the keratinocytes was added to the kit coated with a primary antibody specific to each protein, and reacted for 1 to 2 hours. Then, the kit was washed, and reacted with a secondary antibody, and the optical density was measured at 450 nm. For quantitative analysis, a reference protein solution for each protein was subjected to the same method as described above to obtain a standard curve, and a sample was quantified based on the standard curve.

As a result, it was revealed that IL-1 alpha, VEGF and FGF were expressed at concentrations of 4068.6 pg/ml, 82.2 pg/ml and 301.3 pg/ml, respectively, in the keratinocyte extract according to one exemplary embodiment of the present disclosure, as shown in FIG. 5.

### Example 3

### Experiment of Wound Healing Effect of Dressing

To measure a wound healing effect of the biological dressing according to exemplary embodiments of the present disclosure, a mouse wound model was used. Mice that were 8 weeks old and weighed 29 to 33 kg on average were subjected to general anesthesia by administering zoletil intraperitoneally to the abdomens of the mice at a concentration of 1 ml/kg. Hair was removed from the dosal regions of the mice, which were disinfected with 70% alcohol. Each of two wounds with the size of 1 cm² was created on the left and right backs of each mouse, respectively. Each of two wounds with the size of 1 cm² was created on the left and right backs of each mouse, respectively. The dressing forming a complex with keratinocytes and 1.5% aqueous alginate solution prepared in Example 1 was applied to wound. Changes in wound size for 2 weeks were measured, and histological analysis was performed. In the case of histological analysis, the removed tissue was embedded in a paraffin block, and microtomed into slices having a thickness of 4 μm. Then, each of the paraffin slides was stained with a Hematoxylin-Eosin stain and a Masson's trichrome stain for analyzing collagen synthesis.

As the control, a wound was covered with gauze, and dressed. Thereafter, changes in wound size were measured and histological anatomies were carried out in the same manner as described above.

Wound healing rate (%) was calculated as a ratio of a healed wound size against total wound size when it is assumed that an area of the wound site on the onset of wound induction is 100%.

The wound healing rate was calculated using the following equation.

Wound healing rate (%)=Area of wound on each measured day (cm²)/Area of wound on wound-induced day (cm²)×100

As a result, as shown in FIG. 6, it could be seen that the wound healing rate was significantly improved when treated with the dressing forming a complex with 1×10⁴ or 1×10⁵ keratinocytes, compared to when treated with the control.

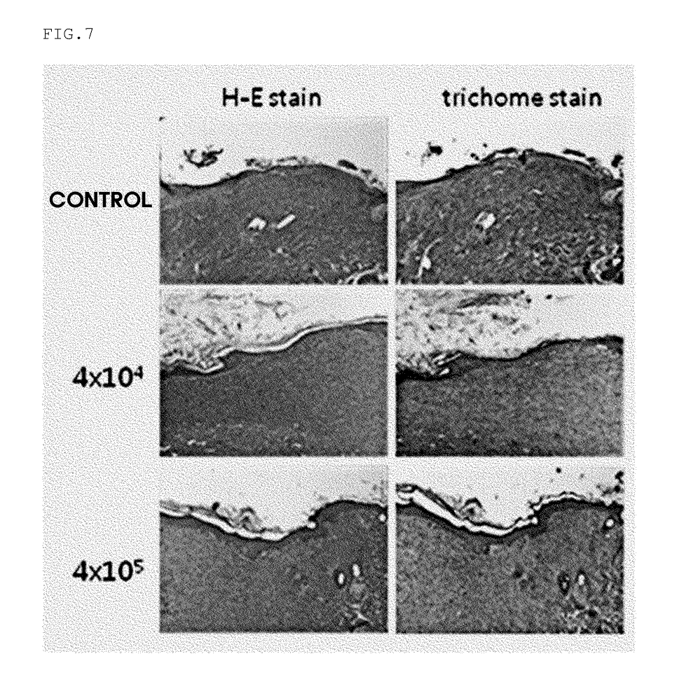

Histological analysis was carried out using a Hematoxlin-Eosin staining method and a Masson's trichrome staining method used to analyze collagen synthesis. As a result, it could be seen that the collagen was synthesized and the skin tissues were healed, as shown in FIG. 7.

### INDUSTRIAL APPLICABILITY

The dressing for treating a wound according to exemplary embodiments of the present disclosure can be useful in maintaining a moisture environment at a wound site using the biocompatible polymer scaffold, and effectively promoting healing of a wound by various growth factors secreted by the skin cells or stem cells attached to the biocompatible polymer scaffold as well.

While exemplary embodiments have been shown and described above, it will be apparent to those skilled in the art that modifications and variations could be made without departing from the spirit and scope of the present disclosure as defined by the appended claims.

1. A dressing comprising:
a biocompatible polymer scaffold; and
skin cells or stem cells attached to the biocompatible polymer scaffold.

2. The dressing of claim 1, wherein the biocompatible polymer scaffold comprises at least one selected from the group consisting of polyvinyl alcohol (PVA), polyurethane (PU), polyethylene (PE), polyacrylic acid (PAA), polyoxyethylene (POE), polyethylene oxide (PEO), polytetrafluoroethylene (PTFE), polypropylene (PP), polyethylene terephthalate (PET), polyamide (PA), polyacrylonitrile (PAN), polyester (PES), polyvinyl chloride (PVC), polyvinylidenefluoride (PVDF), polysiloxane (a silicone rubber), polyglycolic acid (PGA), polylactic acid (PLA), polymethacrylic acid (PMA), polyacrylamide (PAM), polysaccaride (PS), polyvinylpyrrolidone (PVP), silicone, alginic acid, sodium alginate, cellulose, pectin, chitin, chitosan, gelatin, collagen, fibrin, hyaluronic acid, natural rubber, and synthetic rubber.

3. The dressing of claim 1, wherein the skin cells are keratinocytes, melanocytes, endothelial cells, hair follicle stem cells, or fibroblasts.

4. The dressing of claim 1, wherein the stem cells are embryonic stem cells, or adult stem cells.

5. The dressing of claim 1, wherein the biocompatible polymer scaffold is coated with a cell-adhesive polymer before the skin cells or stem cells are attached to the biocompatible polymer scaffold.

6. The dressing of claim 5, wherein the skin cells or stem cells are cultured after the skin cells or stem cells are attached to the biocompatible polymer scaffold.

7. The dressing of claim 1, wherein at least one surface of the biocompatible polymer scaffold to which the skin cells or stem cells are attached is coated with the cell-adhesive polymer.

8. The dressing of claim 7, wherein the skin cells or stem cells are cultured after the biocompatible polymer scaffold is coated with the cell-adhesive polymer.

9. The dressing of claim 1, wherein the skin cells or stem cells attached to the biocompatible polymer scaffold are cultured.

10. The dressing of claim 5, wherein the cell-adhesive polymer is alginic acid, fibrin, gelatin, collagen, or hyaluronic acid.

11. A method for preparing a dressing, comprising:
coating a biocompatible polymer scaffold with a cell-adhesive polymer; and
attaching skin cells or stem cells to the biocompatible polymer scaffold coated with the cell-adhesive polymer.

12. The method of claim 11, further comprising
culturing the skin cells or stem cells attached to the biocompatible polymer scaffold.

13. A method for preparing a dressing, comprising:
attaching skin cells or stem cells to a biocompatible polymer scaffold; and
coating the biocompatible polymer scaffold having the skin cells or stem cells attached thereto with a cell-adhesive polymer.

14. The method of claim 13, further comprising
culturing the skin cells or stem cells after the polymer scaffold is coated with the cell-adhesive polymer.

15. A method for preparing a dressing, comprising:
preparing a mixed solution by mixing skin cells or stem cells with a cell-adhesive polymer; and
attaching the mixed solution to a biocompatible polymer scaffold.

16. The method of claim 15, further comprising
culturing the skin cells or stem cells after the mixed solution is attached to the biocompatible polymer scaffold.

17. The method of claim 11, wherein the biocompatible polymer scaffold comprises at least one selected from the group consisting of polyvinyl alcohol (PVA), polyurethane (PU), polyethylene (PE), polyacrylic acid (PAA), polyoxyethylene (POE), polyethylene oxide (PEO), polytetrafluoroethylene (PTFE), polypropylene (PP), polyethylene terephthalate (PET), polyamide (PA), polyacrylonitrile (PAN), polyester (PES), polyvinyl chloride (PVC), polyvinylidenefluoride (PVDF), a polysiloxane (a silicone rubber), polyglycolic acid (PGA), polylactic acid (PLA), polymethacrylic acid (PMA), polyacrylamide (PAM), polysaccaride (PS), polyvinylpyrrolidone (PVP), silicone, alginic acid, sodium alginate, cellulose, pectin, chitin, chitosan, gelatin, collagen, fibrin, hyaluronic acid, natural rubber, and synthetic rubber.

18. The method of claim 11, wherein the skin cells are keratinocytes, melanocytes, endothelial cells, hair follicle stem cells, or fibroblasts.

19. The method of claim 11, wherein the stem cells are embryonic stem cells, or adult stem cells.

20. The method of claim 11, wherein the cell-adhesive polymer is alginic acid, fibrin, gelatin, collagen, or hyaluronic acid.
